(19) United States
(12) Patent Application Publication
(10) Pub. No.: US 2025/0093317 A1
(43) Pub. Date: Mar. 20, 2025

(54) AIR QUALITY MONITORING SYSTEM AND METHOD

(71) Applicant: BTS KURUMSAL BILISIM TEKNOLOJILERI ANONIM SIRKETI, Istanbul (TR)

(72) Inventors: Gökhan YURDAKUL, Istanbul (TR); Berk CANBERK, Istanbul (TR); Yagmur YIGIT, Istanbul (TR); Khayal HUSEYNOV, Sisli, Istanbul (TR); Özgür PALANTÖKEN, Istanbul (TR)

(21) Appl. No.: 18/559,596

(22) PCT Filed: Dec. 29, 2022
(86) PCT No.: PCT/TR2022/051669

§ 371 Date: Nov. 8, 2023

(30) Foreign Application Priority Data
Dec. 27, 2022 (TR) 2022/020742

(51) Int. Cl. G01N 33/00; H04W 4/38

(52) U.S. Cl. CPC G01N 33/0004; H04W 4/38

(57) ABSTRACT

Disclosed is an air quality monitoring system and method that uses the Industrial Internet of Things (IIoT) and Digital Twin (DT) technologies and creates a YANG (Yet Another Next Generation) based data model, resulting in a low round-trip time, high DT synchronization, and low DT latency.

### TECHNICAL FIELD

The invention relates to an air quality monitoring system and method that uses the Industrial Internet of Things (IIoT) and Digital Twin (DT) technologies and creates a YANG (Yet Another Next Generation) based data model, resulting in a low round-trip time, high DT synchronization, and low DT latency.

### PRIOR ART

Data collection is an important part of sensor devices, related technologies such as IIoT (Industrial Internet of Things) and Wireless Sensor Networks (WSN). A study in the literature conducted a comprehensive review of data collection in the IIoT, WSN, and sensor cloud research areas, and its findings reveal that over the past five years, current data collection studies have generated results that are largely consistent and stable. In one of the existing applications, a light-weight approach leveraging the recent development of IPv6-based open standards for accessing wireless resource constrained networks is proposed to enable device management of wireless sensor devices. A prototype was developed to test the performance of the proposed approach in managing wireless sensor devices, thus contributing to the development of IIoT data collection.

Another study in the literature proposes an intelligent data collection technique to identify energy-aware disjoint dominating sets that operate as data collection nodes at each round to improve WSN lifetime. This study primarily concentrated on energy conservation to ensure the effective functioning and longevity of WSNs. The effectiveness of the proposed technique has been demonstrated mathematically and in simulation. The subject of another study is the IIoT testbed with a security-oriented smart airport cyber twins. The solution consists of numerous heterogeneous IIoT devices and communication protocols that can be used for automatic interconnection and remote access. A data management system that dynamically gathers, evaluates, and classifies IIoT telemetry data received from the network is proposed. In a study, a privacy-preserving data collection and computation offloading scheme is proposed for efficient and secure big sensor data collection in fog computing assisted IoT. The simulation results show that the proposed method is an efficient data collection, computation offloading scheme with strong privacy protection. In one of the existing studies, concurrent data collection trees were designed for IoT applications. Their findings show that the proposed tree structures perform better in data collection processes.

Some studies in the literature focus on air quality monitoring using DT (Digital Twin) technology. In one of the studies, a detailed review of existing DT research in the field of smart cities was made and also its relationship with Industry 4.0 applications was examined. In this study, the importance of pollution monitoring in the smart city field is emphasized and current pollution monitoring studies are reviewed. The current challenges of city-scale DTs are also addressed. In another study, a Zurich city-scale DT is presented to facilitate use in various applications such as air and noise pollution monitoring. A study is presented that supports the active digital participation of citizens in urban planning procedures by visualizing various data covering various applications such as thermal monitoring of the city. Attention was drawn to the importance of using open spatial data in promoting the dissemination of DT at the city scale and developing new applications. Another study discusses a new strategy for producing geospatial data as well as a three-dimensional city model of Vienna that fundamentally rethinks geospatial data in geodata processes. After mentioning the effect of the level of detail of the three-dimensional city representation, the results of the simulation calculating the pollution distribution within a city are presented. In another study, a city-scale DT prototype is presented to combat congestion, air pollution, growth management and the limited capacity of the local energy infrastructure problems in the Cambridge city area. The importance of DT was emphasized at the city scale to reflect the characteristics of the urban and socio-political context. A study proposed a federated learning-based air-to-ground air quality sensing framework for fine-grained three-dimensional air quality monitoring and forecasting. In addition, a graph convolutional neural network-based extended short-term memory model is proposed for ground sensing systems. In general, most current air pollution monitoring DT studies focus on smart city domain, particularly DT at the city scale.

The volume of data processed is rising quickly in tandem with technological innovation. Therefore, for certain applications such as air quality monitoring, anomaly detection, etc., only necessary data should be processed instead of processing all data in order to use the relevant systems more efficiently. Thus, the overload in the system is reduced. Efficiency in data gathering is crucial for IIoT systems since the decision-making process can be significantly impacted by the up-to-dateness of the data acquired. In addition, efficient IIoT data collection reduces energy consumption, latency, network lifetime and overall cost. None of the previous studies specifically covered IIoT-based air quality monitoring in terms of data collection. Therefore, the performance of existing air quality monitoring systems is relatively low. Current air pollution monitoring studies have high round-trip time (RTT), fairly low DT synchronization, and relatively high DT latency. Therefore, current IIoT-based air quality monitoring efforts are inefficient.

During the research carried out in the current art, the application with number CN214334880U was found. The application relates to the air quality forecasting model. The model makes use of temperature and environmental data from the immediate vicinity of the city. However, the application does not mention the use of Industrial Internet of Things (IIoT) and Digital Twin (DT) technologies.

As a result, due to the negativities described above and the inadequacy of the existing solutions on the subject, it was necessary to make an improvement in the relevant technical field.

### Purpose of the Invention

The invention seeks to address the aforementioned drawbacks and is motivated by current events.

The main purpose of the invention is to improve data collection performance between DT and brain layers by developing a YANG (Yet Another Next Generation) based data model using Industrial Internet of Things (IIoT) and Digital Twin (DT) technologies. As a result, lower Round-Trip Time (RTT), higher DT synchronization and lower DT delay are provided in the interaction between DT and brain layers.

Air quality monitoring systems have spatial and temporal complexity. Spatial complexity affects how much memory the system uses. This complexity can be minimized by only pulling the relevant data into the air quality monitoring module. Temporal complexity is related to end-to-end latency. This complexity should be reduced to improve the service quality of the system. Fine-grained air quality monitoring is possible with the invention's subject. By preventing the system from being overloaded, the calculation time in the system is reduced. Thus, the operation of the proposed system becomes more efficient and its spatial and temporal complexity is reduced.

In order to fulfill the objectives described above, the invention is an air quality monitoring system, the physical network layer where the physical objects in a city are located, the sensor that collects and transmits data on all relevant parameters in the city, the IIoT gateway that receives the data from the sensor and provides the interaction between the physical objects and the digital twin, digital twin layer that bridges the gap between physical and digital domains by creating a real-time copy of the physical network, YA-DA modeling unit with YANG-based data model using IIoT and Digital Twin technologies, in which air quality key performance indicators are defined, interface between the digital twin layer and the brain layer, the brain layer that receives the required data through the YA-DA modeling unit and interface, and monitoring module where the examined data is displayed on the air quality in the relevant region.

The structural and characteristic features of the invention and all its advantages will be understood more clearly thanks to the FIGURE given below and the detailed description written with reference to these FIGURE, and therefore the evaluation should be made by taking these FIGURE and detailed explanation into account.

### FIGURES TO HELP UNDERSTANDING OF THE INVENTION

FIG. 1, is the representative block diagram view of the system which is the subject of the invention.

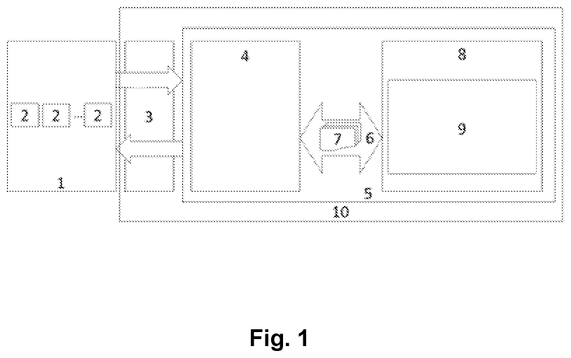

### DESCRIPTION OF PART REFERENCES

1. Physical network layer
2. Sensor
3. IIoT gateway
4. Digital twin layer
5. Digital twin network
6. Interface
7. YA-DA modelling unit
8. Brain layer
9. Monitoring module
10. Cloud

### DETAILED DESCRIPTION OF THE INVENTION

In this detailed explanation, the preferred embodiments of the air quality monitoring system and method, which are the subject of the invention, are explained only for a better understanding of the subject.

The subject of the invention, in general, includes.
transmission of data collected via sensors (2) located in the physical network layer (1) to the IIoT gateway (3),
the IIoT gateway (3) receives the data from the sensor (2) and transfers the data to the digital twin layer (4) by providing the cyber-physical interaction between the physical objects and the digital twin layer (4),
creation of a real-time copy of the physical network at the digital twin layer (4),
synchronization and matching of the digital twin layer (4) with the physical network layer (1), which consists of physical objects,
using IIoT and Digital Twin technologies and having a YANG-based data model, the YA-DA modeling unit (7) determines the required data using air quality performance indicators and transmits it to the brain layer (8),
brain layer (8) analysing the data and displaying the air quality via the monitoring module (9),
the scaling process steps of digital twin network (5) system requirements located in the cloud (10).

The physical network layer (1) is the network of physical objects in a city. Data regarding all relevant parameters in smart cities are collected by means of sensors (2). The IIoT gateway (3) receives data from the sensor (2) and provides the interaction between the physical objects and the digital twin. In digital twin technology, digital copies of physical objects are created in the digital twin network. In order for digital copies to have the same properties as the physical object, an interaction (data transfer and synchronization) is required between them.

The digital twin layer (4) bridges the gap between the physical and digital domains by creating a real-time copy of the physical network. The digital twin network (5) is the network formed by the digital twin layer (4) and the brain layer (8). The communication between the digital twin layer (4) and the brain layer (8) takes place through the interface (6). In the brain layer (8), the data is analyzed and the air quality is visualized in the system.

YA-DA modeling unit (7) is a YANG-based data model with tree data structure that performs better in IIoT data collection. The proposed system is developed using IIoT and DT (Digital Twin) technologies.

The brain layer (8) receives only the required data via the YANG-based YA-DA modeling unit (7) and the interface (6). Within the YA-DA modeling unit (7), the air quality key performance indicator (KPI) is defined. The air quality sensors to be used in the system and the data to be obtained from these sensors (CO, NO, etc.) are defined. KPIs are created based on the information in this data. Thus, the brain layer (8) receives only the required data via the YANG-based YA-DA modeling unit (7) and the interface (6). The air quality data collected are displayed via the brain layer (8). There are international threshold values determined for air quality. The brain layer (8) compares the data it receives with predetermined threshold values and analyses it.

The air quality in the relevant area is monitored by the monitoring module (9). The cloud (10) is the online computing resource where the digital twin network (5) is located and can be scaled at any time. The digital twin layer (4) and the brain layer (8) are situated within the digital twin network (5) located in the cloud (10), scaling the system requirements.

YANG (Yet Another Next Generation) is a data modeling language for configuration and monitoring. The hierarchical organization of data is modeled in YANG as a tree in which each node has a name-value pair or a set of child nodes. It provides clear and concise descriptions of nodes along with the interaction between nodes. A YANG module contains a combination of related definitions. It can import definitions from external modules and contain comments from submodules. Also, the hierarchy can be extended by allowing a module to add data nodes defined in another module. Taking advantage of the YANG model, the YA-DA modeling unit (7) was created to improve IIoT data collection performance. YA-DA the modeling unit (7) provides better performance in IIoT data collection by being developed in a tree data structure.

In the YA-DA modeling unit (7), four main data node types are defined: leaf nodes, leaf list nodes, container nodes, and list nodes:
Leaf Node contains at most one instance in the data tree. A leaf has a value and has no child nodes.
Leaf List Node defines a set of uniquely identifiable nodes rather than a single node. Each node has a value but no child nodes.
Container Nodes are used to group related nodes into a subtree. A container node has no value, but rather a set of child nodes of any type, such as leaves, lists, container nodes, leaf lists.
List Node defines a series of list entries. Each entry is defined like a container node and is uniquely identified by key leaves. A list can define multiple key leaves and contain any number of child nodes of any type, including leaves, lists, and container nodes.

The data collected through the sensors (2) located in the physical network layer (1) are transmitted to the IIoT gateway (3). The IIoT gateway (3) transfers the data it collects to the digital twin layer (4) located in the digital twin network (5) located in the cloud (10). The physical network layer (1) is synchronized with the digital twin layer (4).

The subject of the invention consists of a physical network layer (1) containing physical objects and a digital twin network (5) comprising a digital twin layer (4) and a brain layer (8). Said digital twin network (5) is located in the cloud (10).

A copy of the physical objects and synchronization between the physical objects and the digital twin layer (4) are created. The IIoT gateway (3) collects the sensor (2) data and provides the cyber-physical interaction between the physical objects and the digital twin (4). Then, the sensor (2) data is received by the digital twin layer (4).

Since the amount of sensor (2) data is more than desired and needed, the system may become very slow. To solve this problem, only necessary data needs to be transferred to the module. Therefore, only the desired data is drawn to the air quality monitoring module (9) in the brain layer (8) via the interface (6), thanks to the air quality key performance indicators defined in the YA-DA modeling unit (7).

1. An air quality monitoring system comprising:
a physical network layer where physical objects in a city are located;
a sensor that collects and transmits data on all relevant parameters in the city;
an IIoT gateway that receives the data from the sensor and provides data transfer and synchronization between physical objects and digital twin;
a digital twin layer that creates a real-time copy of the physical network;
a digital twin network formed by the digital twin layer and a brain layer;
a YA-DA modeling unit, which has a YANG-based data model using IIoT and the digital twin technologies, in which air quality key performance parameters are defined;
an interface that provides the communication between the digital twin layer and the brain layer;
wherein the brain layer receives the required data via the YANG-based YA-DA modeling unit and interface and compares the received data with predetermined threshold values; and
a monitoring module where the compared data are displayed on the air quality in the relevant region.

2. The air quality monitoring system according to claim 1, wherein the digital twin layer and the brain layer are located within the digital twin network placed in the cloud, and scale the system requirements.

3. An air quality monitoring method, comprising the process steps of:
transmission of data collected via sensors located in a physical network layer to an IIoT gateway;
the IIoT gateway receives the data from the sensor and transfers the data to a digital twin layer by providing cyber-physical interaction between the physical objects and the digital twin layer;
creation of a real-time copy of the physical network at the digital twin layer;
synchronization and matching of the digital twin layer with the physical network layer, which comprises physical objects;
using IIoT and Digital Twin technologies and having a YANG-based data model, a YA-DA modeling unit determines the required data using air quality performance indicators and transmits to the brain layer;
data analysis of the brain layer and display of air quality via a monitoring module.

4. The air quality monitoring method according to claim 3, comprising examining the air quality through air quality monitoring module and the data modeling of the YA-DA modeling unit in the digital twin network located in the cloud.
